(19) United States
(12) Patent Application Publication
(10) Pub. No.: US 2024/0269930 A1
(43) Pub. Date: Aug. 15, 2024

(54) ADDITIVE MANUFACTURING USING LOW VISCOSITY MATERIALS

(71) Applicant: Copner Biotech Ltd, Abergavenny, Monmouthshire (GB)

(72) Inventors: Jordan C. COPNER, Abergavenny, Monmouthshire (GB); Alan J. COPNER, Abergavenny, Monmouthshire (GB)

(21) Appl. No.: 18/566,850

(22) PCT Filed: May 9, 2022
(86) PCT No.: PCT/GB2022/000051

§ 371 Date: Dec. 4, 2023

(30) Foreign Application Priority Data
Jun. 6, 2021 (GB) 2108078.3

(51) Int. Cl. B29C 64/336; B29C 64/112; B29C 64/386; B29C 64/40; B33Y 10/00; B33Y 50/00; B33Y 70/00

(52) U.S. Cl. CPC B29C 64/336; B29C 64/112; B29C 64/386; B29C 64/40; B33Y 10/00; B33Y 50/00; B33Y 70/00

(57) ABSTRACT

Disclosed herein is a process for 3D printing low viscosity material to produce a 3D printed article, the process comprising the steps of: a) providing software for moving a printing head relative to the article being printed; b) moving the print head relative to the article being printed according to the software, such that the article is printed in a layer by layer manner in multiple layers each having an article area; c) for at least some of the layers, printing a first material only to a first part of the article area, and printing a second material only to a second part of the article area different to the first area; and wherein, the first material is self-supporting to provide an enclosure for the second material which is a low viscosity, substantially non self-supporting, material when printed initially, which is preferably a self-assembling material forming a self-supporting article after maturing, so the first supporting material can then be removed if required.

This invention relates to methods for 3D printing low viscosity materials with high precision, including, but not limited to 3D printing of self-assembling polymeric materials such as self-assembling gels, by material deposition from a dispensing nozzle. That 3D printing technique is referred to in a prior related patent application GB2108078.3 as ‘inkjet’ printing because it resembles printing a jet of ink.

3D printing is an established method of manufacture. Whilst there are many types of 3D printing, they all revolve around the same concept of fabricating a specific design defined by a data set, in a layer-by-layer fashion. This concept is often known as sequential additive manufacturing but is better known as 3D printing.

Classically 3D printing is achieved by material extrusion using a fused deposition modelling (FDM) 3D printer. Therein, typically, material is forced from a nozzle in a molten state and solidifies during cooling or is cured by UV light, fusing it to adjacent material, often the underlying layer. To extend this type of process to printing of low viscosity materials would have tangible benefits of speed of print and more versatility of material that could be printed; but with the obvious issue of material overspray over already printed lower structure layers. Previous attempts at solving this problem are based on finding low viscosity materials that can very quickly solidify such as UV polymerizable inks. However, in most cases low viscosity materials are considered to be non-printable by conventional 3D printers. Moreover, in some industries, such as biotechnology, it is not possible to change significantly the mechanical properties of the low viscosity material or expose it to strong light energy, and so those materials have not been capable of being 3D printed.

EP 3351366 and similar application EP3560683 describe the use of multiple inkjet nozzles for printing low viscosity curable materials but these are of little use in the biotechnological field for the reasons mentioned above, and principally because the use of a strong stereolithographic light energy source for curing the printed materials will not be suitable for the materials proposed in this application.

WO2020257669 describes multiple inkjet nozzles to print the component layers of a PCB board at different print stations. The materials described in the application are not the same as the materials described herein.

So, the inventors have devised a process capable of 3D printing low viscosity material with high accuracy of print dimensions. That process employs a 3D printer performing at least two print passes for some layers printed, whereby each of the at least two passes lay down a different material. The first material print pass(es) provides an enclosure or mould area, referred to in said previous related application as a ‘template negative space/void mould’ to be in-filled by the second subsequent print pass of low viscosity material. It is envisaged that the second material will not, at least initially after printing be substantially self-supporting, but will have at least some inherent strength immediately at printing, and enough strength to support itself for one printed layer at least without needing a surrounding mould first material layer. So, whilst the preferred option is for a supporting first material to be laid down, it need not be laid down first for every layer. In a refinement, multiple mould areas can be formed by the first material for multiple discrete fills of the second low viscosity material, or vice versa. On completion of each layer the print passes provide a generally flat layer surface ready to accept the next print layer. This arrangement resolves the issue of material overspray between print layers, and, if the shape of the construct demands it, the low viscosity material can be overprinted by the first material supported by floating on the second material.

In a further refinement, the second material can be a self-assembling polymer such as one or more of: collagen types 1 to 28, jellyfish collagen, nascent protein polypeptides, deoxyribonucleic acids (DNA) or ribonucleic acids (RNA). The first printed material can be a biocompatible, but possibly ultimately sacrificial, material such as one or more of gelatin, alginate, or thermo-responsive hydrogels. Material(s) employed for the print pass intended for the mould area, if sacrificial in nature, can be removed after the self-assembling second material has set (possibly over a few hours or days); thereby exposing the required 3D article's shape formed purely from a low viscosity self-assembled polymer material ready for use, for example possibly now with internal voids vacated by the sacrificial material, to accept nutrients during cell culture. Removal of the first material can be on completion of the print run of all layers, or even when the second material article is to be used, so then the first material acts as a mould in the first instance, and then also as transit packaging.

The invention extends to any combination of features disclosed herein, whether they are mentioned in combination herein or not.

The invention can be put into effect in numerous ways, illustrative embodiments of which are described below with reference to the drawings, wherein:

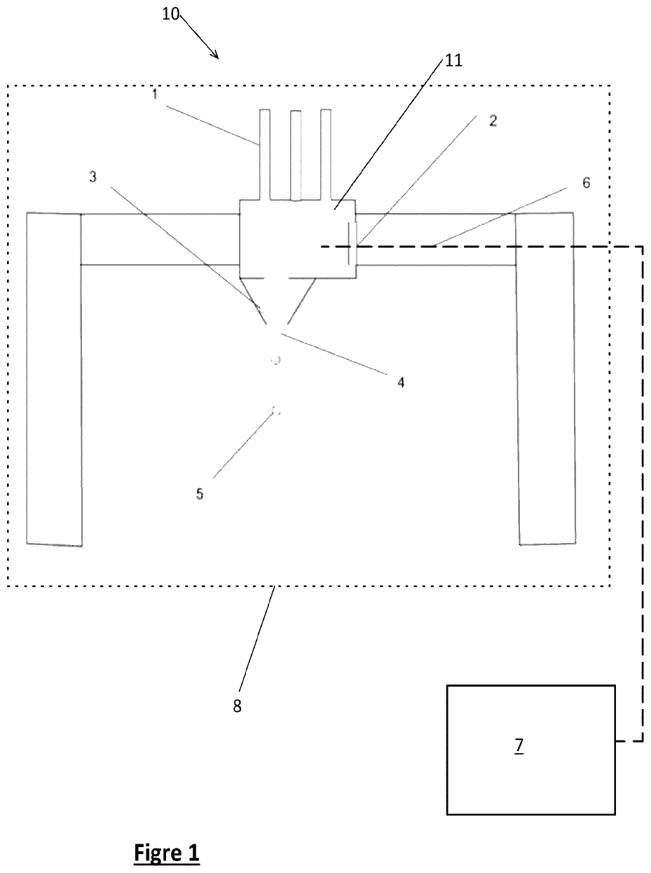

FIG. 1 shows a schematic representation of a 3D printer used to put the invention into effect;

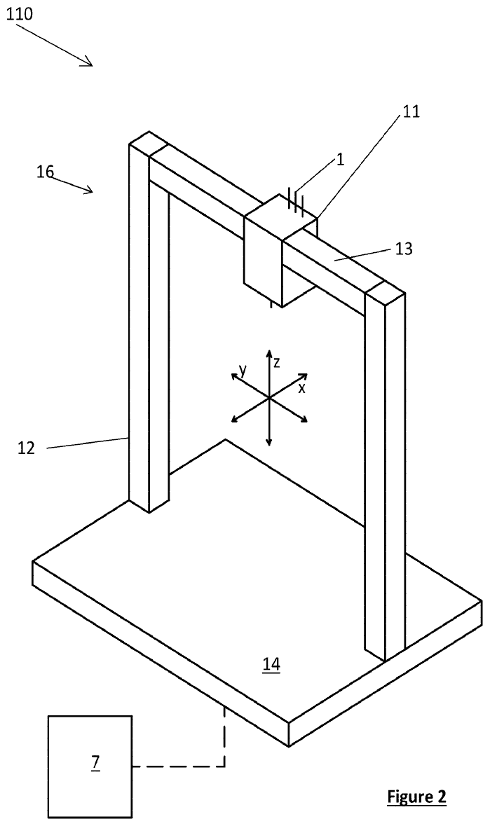

FIG. 2 shows an alternative printer to that shown in FIG. 1;

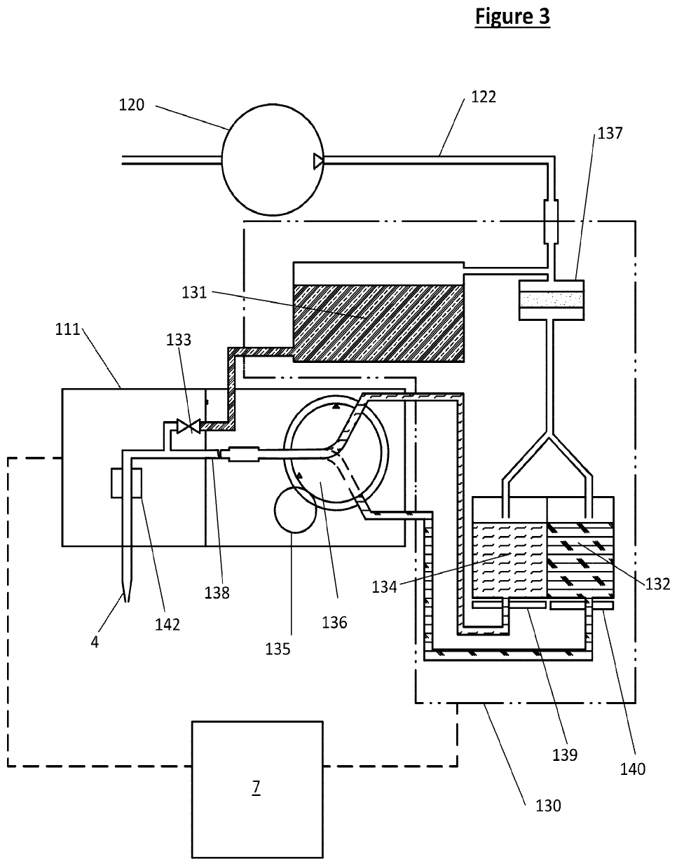

FIG. 3 shows a print formulation cassette and printing head for use with printer of FIG. 1 or 2;

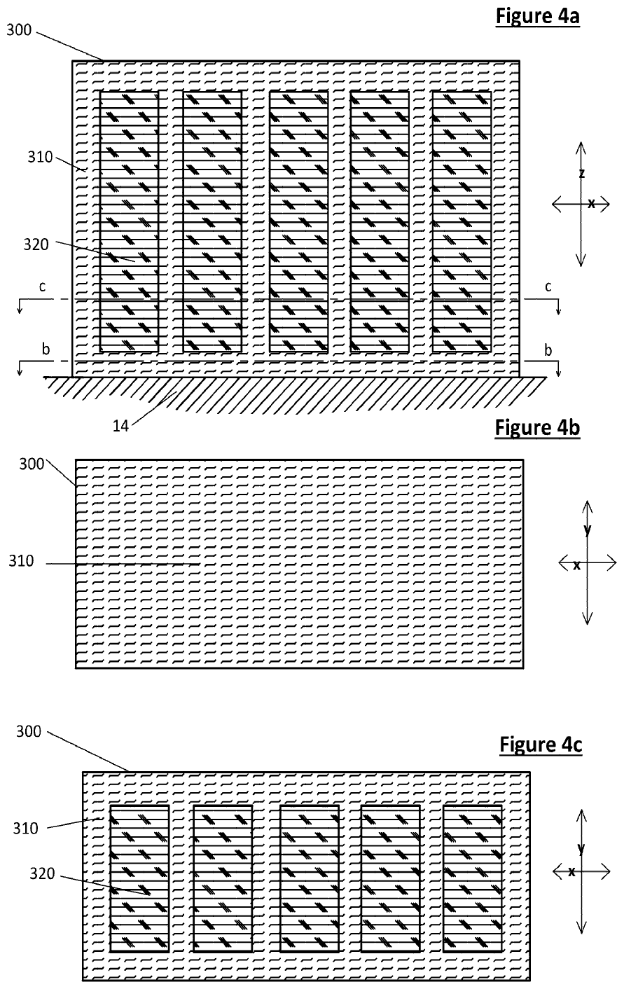

FIGS. 4a, b and c show examples of cross sections of a printed article; and

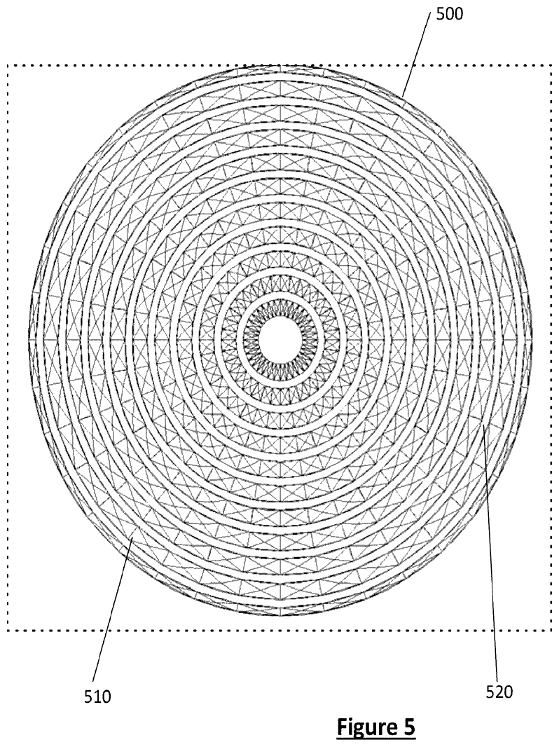

FIGS. 5 to 12 show various examples of articles printed, according to the invention.

The invention, together with its objects and the advantages thereof, may be understood better by reference to the following description taken in conjunction with the accompanying drawings, in which like reference numerals identify like elements in the Figures.

The invention utilises a conventional 3D printer modified as described below. A digital representation of a desired three-dimensional construct or article is produced as a software file, preferably as a Standard Tessellation Language (STL) file although other formats could be employed. The file is converted into instructions suitable for the printer by slicer software which produces representations of the desired article in multiple layers for printing. The printer hardware interprets the sliced STL file to provide commands to move the print head nozzle and/or article and dispense an appropriate material at the right time.

FIG. 1 shows schematically, such a modified printer 10, including a print head 11 having printable material formulation delivery tubes 1 (three in this case), a variable output heating element 2, a print nozzle driver 3, which could be thermal/piezoelectric elements of a commercially available inkjet printer head, a print nozzle 4, for illustration only—material droplets 5 which will be expelled from the print nozzle in use in a controlled manner, and a conventional commercially available robotic 3D printing arm 6, linked to a controller 7. The printer is contained within a controlled environment 8, in this case a sterile and positive-pressure clean environment. Printing is progressed at a relative bed to nozzle velocity of up to 55 mm per second, by is usually about 10 to 50 mm per second FIG. 2 shows a perspective view of a similar printer 110 having the same attributes as the printer 10 mentioned above. A conventional assembly 16 includes two uprights 12 moveable in the x direction on a temperature-controlled printing table 14. The moveable uprights support a horizontal beam 13 moveable in the z direction on the uprights 12. In turn the beam 13 supports a print head 11, itself moveable in the y direction. The printhead 11 has three inputs 1, in this case for two different printing materials and a cleaning fluid, each routed to a print nozzle the same or similar to the print nozzle 4 shown in FIG. 1. The printer 110 will be under the control of a controller 7. The printer 110 can, in practice, be enclosed and controlled in the same manner as described for the printer shown in FIG. 1. In use the print bed 14 is temperature controlled and will be within a temperature-controlled enclosure operated at between 5 and 37 degrees Celsius (degrees C.).

FIG. 3 shows another schematic of another print head 111, and a disposable supply cassette 130, for supplying printable material to the print head 111. In this embodiment a controllable and variable output pneumatic pump 120 forces pressurised air into the cassette 130 via a disconnectable supply tube 122 and a filter 137 when printing is called for by the controller 7. The pump operates at a range of zero to 600 mbar(g) The cassette 130 includes two printable material vessels 132 and 134, each pressurisable by the pump 120. The vessels 132 and 134 are each temperature controlled via independent Peltier elements 139 and 140 respectively, such that the material formulations contained in the vessels can be heated or cooled as needed. It is intended that the vessels can be cooled down to zero degrees C. or warmed, for example ready for printing, to up to 37 degrees C. Temperature monitoring and temperature reporting, along with vessel fill levels can be reported to the controller 7. Fluid outlets in the vessels 132 and 134 lead to the print head 111. Each outlet feeds a respective port of a three-port valve 136, which is operable, e.g., by a rotary electrical actuator 135, to rotate thereby selecting the material of either vessel 132 or 134 as the material to be transferred via output transfer tube 138 to a print nozzle 4 of the printhead 111. The cassette 130 includes also a cleaning fluid reservoir 131, again pressurisable by the pump 120. With the three-port valve 136 in a mid-position so there is no back-flow, an electrically operable cleaning valve 133 can be opened to flush the nozzle 4 with cleaning fluid from the reservoir 131 if it were to block, or at the end of a print pass. An auxiliary heater 142 is provided in the print head to warm the printable materials if needed.

In this invention a series of STL files are used, each representing different areas of a layer to be printed. Each print layer can have two or more print passes of different materials; each print pass being defined by a separate STL file. The materials to be printed pass through the single printer nozzle 4 to maintain spatial accuracy when printing. Depending on the accuracy, print passes of between 10 and 1000 microns in thickness are possible.

FIG. 4a shows a vertical cross section of an example of an article 300 printed according to the invention. The article is formed from a self-supporting material 310, in this case gelatin formed in layers, and an initially low viscosity material 320 in this case jellyfish collagen, which when printed is not substantially self-supporting although it will hold up if one layer is printed. Each volume of material shown has been formed in layers. FIG. 4b is a horizontal cross section on sectional line b-b of FIG. 4a and shows an initial layering of only the self-supporting material 310 which will take place on, for example, the bed 14 shown in FIG. 2 or a non-stick substrate attached to the bed 14. Once enough print passes have been made to build up a suitable layer, for example a 2 mm layer, the printer can lay down another layer of material 310 but this time in an area as shown in FIG. 4c, which is a section on the sectional line c-c shown in FIG. 4a. This print pattern leaves out the areas with different shading and thereby forms a void or mould area for accepting the low viscosity material 320. After that initial printed area is completed by a first print pass, the unprinted area is printed in a second pass with the low viscosity material 320, which is contained by the previously printed void/mould area left unprinted by the first pass of material 310. The printing is repeated to obtain a 3D article having the desired shape and volume formed, in this case, from materials 310 and 320. A cap similar to the base construction shown in FIG. 4b can be printed over the layers shown in FIG. 4c, to encapsulate the material 320, and arrive at the final cross section shown in FIG. 4a. The self-assembling material 320, over time will assemble into a useable article which eventually does not then need its supporting material 310, which can be removed.

Prior to each print pass, the printer nozzle 4 will be driven to a predefined location by software of controller 7, to go through a material charge cycle. This involves printing a set amount of the required print material into a removable waste receptable.

On completion of each print pass, the printer nozzle 4 will be driven to a further predefined location to go through a cleaning cycle. This cleaning cycle involves printing a predetermined amount of the cleaning material into a removable waste receptable.

This practical technique facilitating the 3D printing of low viscosity material/inks/substrates in particular provides tangible accuracy benefits in the 3D bioprinting field.

3D bioprinting according to the invention can be utilised in tissue engineering, pharmaceutical development and cancer research. Practical examples of the invention include:
Bio inks of low viscosity printed into a pre-layered layer of high viscosity gel (such as hydrogel), the bio-ink including concentrated cancer cell suspensions to fabricate cancer cell spheroids in the voids formed by the first material 310. Formation of cancer cell spheroids in this way with a high degree of repeatability would be a valuable tool in cancer research.
Self-assembly polymers used as a mould/void fill material. These polymers will self-assemble and translate into the pre-designed 3D structure if/when the supporting mould material (310) is removed.
Bio-inks used to fill the voids laden with living algae. The resulting 3D construct is a functional algae bioreactor.
Other eucaryotic cells, algae or other bio-viable entities such as bacteria fungi or viruses could to carried in the printed materials, for example used as bio-indicators or biosensors.
Collagen laden bioinks printed into pre-layered high viscosity gel (such as hydrogel). The collagen laden bioink can be precisely deposited into a pre-designed void to encourage the formation of uniform collagen fibril organisation. Uniform fibril formation and organisation will ultimately improve construct structure/microarchitecture and optimise subsequent function, through improved strength, flexibility and longevity of the printed tissue construct. One example application of this approach would be in the formation of radial tie fibres, as well as circumferential collagen fibres bundles in a meniscus of a knee joint. Another example would be the zonal layers of cartilage observed in articular cartilage. A further example would be optimised tendon/ligament constructs with uniform and tightly packed collagen fibril organisation.

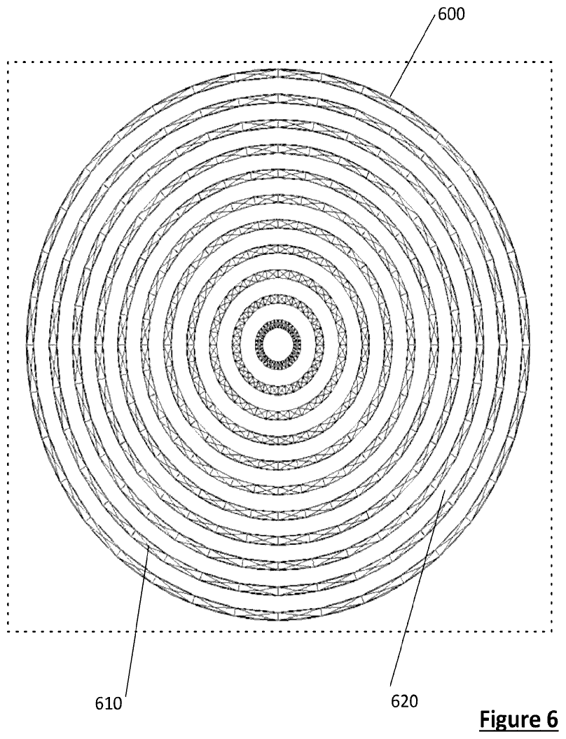

FIG. 5 shows in one embodiment; a simple non-limited example of a top view Concentric Circles model 500 defined in STL for a printing pass to produce a mould template 510 when printed for supporting low viscosity material 520. In an alternate embodiment FIG. 5 shows a simple non-limited example of a top view Concentric Circles model defined in STL for a printing pass to fill alternate negative space/voids realised by the previously printed model material FIG. 6 when printed.

FIG. 6 shows in one embodiment; a simple non-limited example of a top view Concentric Circles model 600 defined in STL for printing pass to produce a material structure fill 610 of the negative space/voids realized by the mould template print pass FIG. 5, of low viscosity material 620 when printed.

In an alternative embodiment FIG. 6 shows a simple non-limited example of a top view Concentric Circles model defined in STL for printing pass to produce a model material layer in which the alternate negative space/voids realised by the model layer print are filled together with the extremities of the model layer print encapsulated by the sacrificial material print pass defined by STL data depicted in FIG. 5.

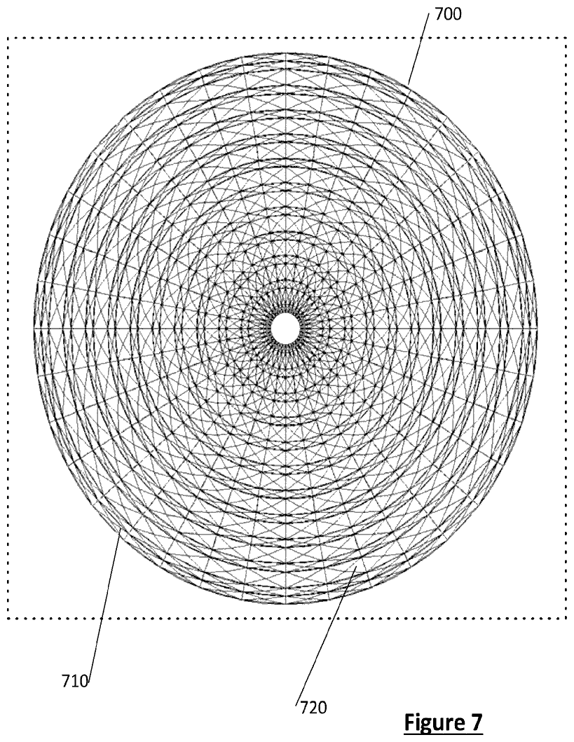

FIG. 7 shows a simple non-limited example of a top view Concentric Circles model 700 defined in STL for layer mould template 710 and filler material structure 720 made by print passes as depicted by FIGS. 5 and 6; showing that these materials will be touching when printed.

Once these print passes have been printed a flat surface is realized for the next layer to be printed on top.

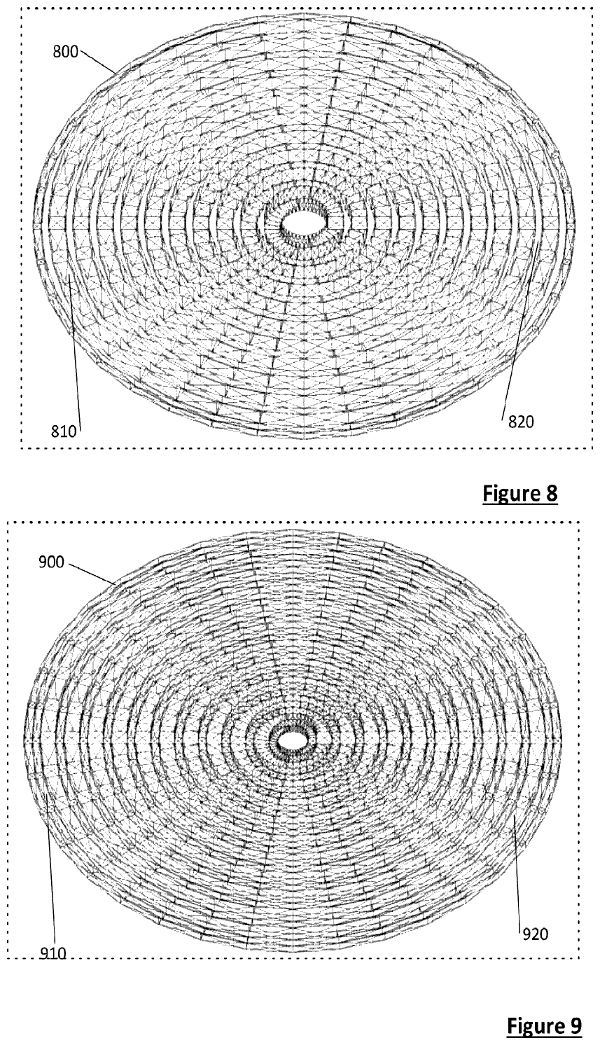

FIG. 8 shows a simple non-limited example of a view (FIG. 5) rotated 45 degrees Concentric Circles model 800 defined in STL for printing to produce a layer mould template 810 and model filler 820 when printed.

FIG. 9 shows a simple non-limited example of a view (FIG. 7) rotated 45 degrees of Concentric Circles model 900 defined in STL to produce layer mould template 910 and model filler 920 together with model material print passes when printed. Once these print passes have been printed a flat surface is realized for the next layer to be printed on top.

FIG. 10 shows in one embodiment; a simple non-limited example of a view rotated 45 degrees of a Concentric Circles model 1000 defined in STL for printing to produce first layer comprising of mould template 1010 filled by structure material 1020, with second layer to produce another mould template, demonstrating void spaces now realized to accept the next material structure fill print for this second layer.

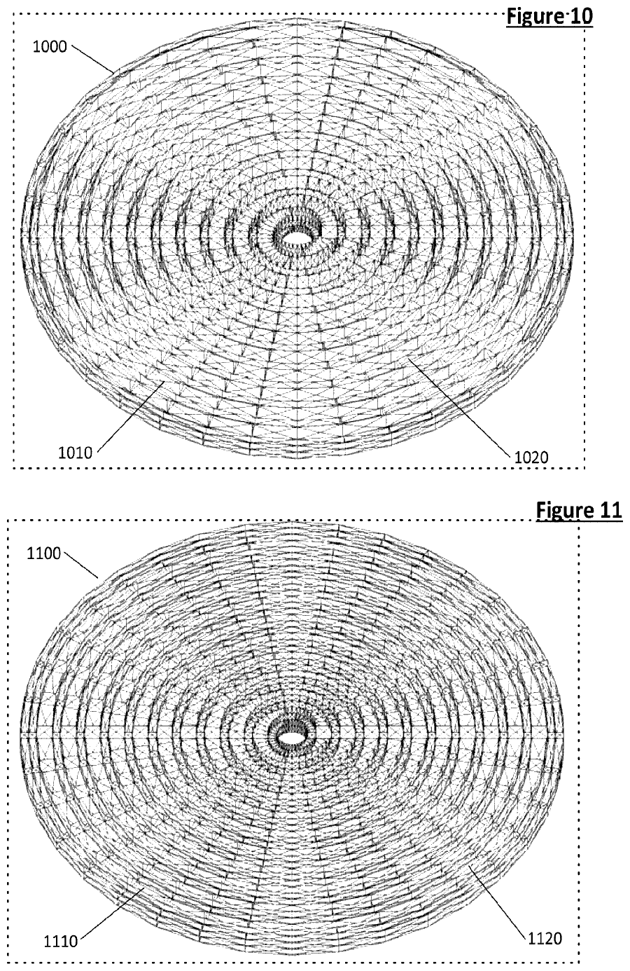

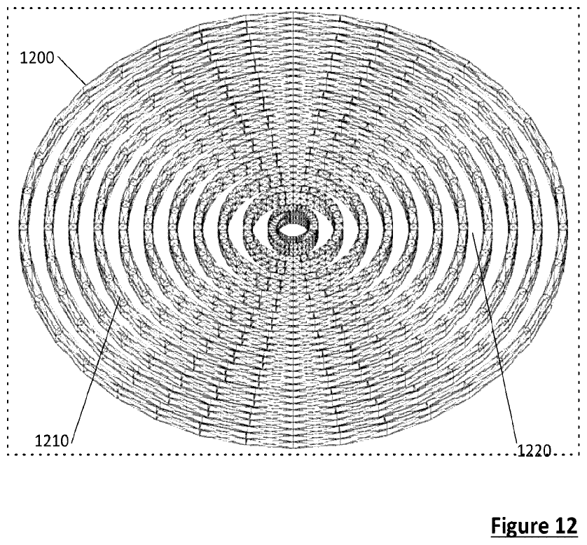

FIG. 11 shows in one embodiment; a simple non-limited example of a view rotated 45 degrees of a Concentric Circles model 1100 defined in STL for printing to produce two print layers, with each layer comprising of mould template 1110 optionally of the same or different materials filled by optionally the same or different model structure material(s) 1120. Once these layers have been printed a flat surface is realized for the next layerto be printed on top.

FIG. 12 shows in one embodiment; a simple non-limited example of a view rotated 45 degrees of a Concentric Circles model 1200 comprising of two layers of model material(s) structure 1210; demonstrating the realization of the required 3D model material(s) structure 1220, once the template mould material(s) layers have been removed post print.

It will be apparent that the invention can be put into effect in numerous ways with only a limited number of examples being given above. For ease of understanding FIGS. 1 and 2 depict the use of commercially available printer heads being of the thermal or piezoelectric variety, but any printer that can expel the desired materials in the correct spatial position will suffice. So non-Cartesian machines could be employed. The delivery system shown in FIG. 3 is just an example. It may be that the materials to be printed could be housed in simple vials in a holder alongside the printer and no dedicated cassette 130 of the type shown is required. On the other hand, a more elaborate cassette could be employed housing multiple materials (e.g., up to 13 different materials to be printed), the cassette including its own pump and flow control arrangements. The use of one material delivery nozzle is preferred but multiple nozzles could be used also.

The process of the invention, in one embodiment begins with initial layer print pass(es) for the fabrication of a template with a pre-designed mould region. This template mould could consist of several different materials, including but not limited to a gelatin, a hydrogel, or the like. This template mould provides voids to encapsulate a pre-designed structure/pattern, in the form of a low viscosity ink material. This comes about via the direct deposition of the ink into the voids. The resultant template mould layer and the freshly deposited material ink form a substantially flat surface, then with no voids. The low viscosity material may also be more than one material on each layer or different material(s) at different layers, to form a complex article, for example where different materials carry different cell types in a complex cellular construction.

The process in an alternative embodiment; begins with initial layer print pass(es) for printing the generally non-supporting material before subsequent layer print pass(es) printing the supporting layer around the previously printed generally non-supporting material. In this embodiment the supporting material serves to fill the voids left by the previous non-supporting layer print pass(es). The resultant non-supporting and supporting material print passes again achieve the desired flat surface with no remaining voids. This technique is made possible because the non-supporting low viscosity material will hold up for a height of one print layer at least. Printing the non-supporting material second in one layer, and then first in the next layer saves having to purge the print nozzle between each print layer.

The result of the methods of 3D printing described above is a stack of printed layers with the pre-designed 3D construct, made from low viscosity ink/substrate material(s), trapped inside supporting material(s). It may be that the shape of the layers changes as the stack is printed, for example where first and/or second materials overlap so that the shape of the construct changes for some layers. The layered material(s) encapsulated within the filled voids can be in direct contact with each other through the layers and can therefore undergo further maturation post print. The result being the fusion of optionally some or all of these layered ink-like material(s) and the creation of a 3D article(s). Support material(s) layers that surround the non-supporting material(s) serve as support/packaging, which can be removed via mechanical, temperature or chemical processes, depending on the material(s) employed. In one example, gelatin support material (310) can be melted at relatively low temperatures in excess of 25 degrees C. for removal prior to deployment of the remaining article 320 into its target application, e.g. in vitro research.

Once the printing process is finished for a layer, another layer with (optionally in one embodiment) the same or different mould material, can be printed onto this just printed layer and subsequently filled with same or different ink material. In an alternate embodiment another layer is printed onto the just printed layer whereby the same or different printed model pattern/material is again initially printed before the alternate negative space/voids realised by this printed model pattern is filled by printing optionally the same or different sacrificial material.

The print layer process repeats for all required model layers; in either or a mixture of the above embodiments; until the required article structure is manufactured.

The concept presented herein is a true 3D model printing process employing a single inkjet nozzle/needle through which all printed materials pass in order for all material print passes to be spatially correct for head position and subsequent print accuracy. Additionally, this patent application describes the printing of multiple layers in which each completed printed layer presents a flat layer (not worked post print) for the next layer to print. This arrangement enables complex 3D models to be accurately printed utilising model component layers with no overspray of model/sacrificial materials to the already printed model component layers.

1. A process for 3D printing low viscosity biomaterials to produce a 3D printed article, the process comprising, in any suitable order, the steps of:
a) providing software for moving a printing head relative to the article being printed;
b) moving the print head relative to the article being printed according to the software, such that the article is printed in a layer by layer manner in multiple layers each layer having an article area;
c) for at least some of the layers, printing a first biomaterial only to a first part of the article area, and printing a second biomaterial only to a second part of the article area, the second part of the area being different to the first area and each of said multiple layers providing a substantially flat surface without voids once printed, for accepting a respective subsequent layer;
wherein, the first biomaterial is self-supporting to provide an enclosure for the second biomaterial which is a low viscosity, substantially non self-supporting but self-assembling polymer biomaterial when printed initially.

2. A process according to claim 1, wherein the first and/or second parts of the area are multiple discrete areas.

3. A process according to claim 1 wherein a selection is made between a) the first biomaterial is printed before the second biomaterial for at least some of the printed layers, and b) the second biomaterial is printed before the first biomaterial for at least some of printed layers.

4. A process according to claim 1, wherein the initial, peripheral and final layers of the article are all formed substantially from the first biomaterial so as to encapsulate the second biomaterial.

5. A process according to claim 1, wherein the first biomaterial is selected from a group consisting of: a biocompatible biomaterial such as gelatin, alginate, thermo-responsive hydrogel, and the second biomaterial is a polymer selected from a group consisting of: collagen types 1 to 28, jellyfish collagen, nascent protein polypeptides, deoxyribonucleic acids (DNA) and ribonucleic acids (RNA).

6. A process according to claim 5 wherein the second biomaterial includes bio-materials such as eukaryotic cells including animal cells, plant cells, algae and fungi or other biomaterials such as bacteria or viruses.

7. A process according to claim 1, wherein the first biomaterial is removed from the printed article, optionally by dissolving the first biomaterial preferentially leaving the second biomaterial intact.

8. A process according to claim 1, wherein the first and second biomaterials are printed by means of expelling the respective biomaterials sequentially from one or more printing nozzles, and preferably from the same printing nozzle.

9. A process according to claim 8, wherein said first and/or second biomaterials are supplied to said nozzle(s) under pressure.

10. A process according to claim 1 including the step of flushing or cleaning the nozzle(s) between printing article areas.

11. A 3D printed article formed from at least two biomaterials each printed in multiple layers, the layers each defining an article area, the biomaterials including a first self-supporting biomaterial printed only to a first part of the article area and a second substantially non self-supporting biomaterial printed only to a second part of the article area different to the first area, the first biomaterial forming a mould for the second biomaterial and optionally the second biomaterial being printed before the first biomaterial for some or each respective layer.

12. An article as claimed in claim 11, wherein the first biomaterial is selected from a list including: a biocompatible biomaterial such as gelatin, alginate, and thermos-responsive hydrogel, and the second biomaterial is a self-assembling polymer selected from: collagen types 1 to 28, jellyfish collagen, nascent protein polypeptides, deoxyribonucleic acids (DNA) and ribonucleic acids (RNA).

13. A process according to claim 1, wherein the first and second parts of article area are in the form of concentric rings.

14. A disposable cassette including respective vessels containing the first and second biomaterials defined in claim 5, each biomaterial being arranged for dispensing to a 3D printer.

15. An article according to claim 11 wherein the first and second parts of article area are in the form of concentric rings.
